(19) United States
(12) Patent Application Publication
(10) Pub. No.: US 2016/0346521 A1
(43) Pub. Date: Dec. 1, 2016

(54) BALLOON-OCCLUDED RETROGRADE TRANSVENOUS OBLITERATION CATHETERS AND RELATED SYSTEMS AND METHODS

(71) Applicant: Regents of the University of Minnesota, Minneapolis, MN (US)

(72) Inventors: Jafar Golzarian, Plymouth, MN (US); Sean Lester Moen, Saint Paul, MN (US); Michael Rosenberg, Eagan, MN (US)

(21) Appl. No.: 15/161,860

(22) Filed: May 23, 2016

Related U.S. Application Data
(60) Provisional application No. 62/167,944, filed on May 29, 2015.

(51) Int. Cl. A61M 25/10; A61B 17/12; A61M 25/00

(52) U.S. Cl. CPC A61M 25/10; A61M 25/1025; A61M 25/0026; A61B 17/12109; A61M 2025/105; A61B 2017/1205

(57) ABSTRACT

A multi-lumen catheter defining a longitudinal axis includes an inflation lumen, a plug lumen, and a microcatheter lumen, each defined by the catheter and extending substantially parallel to the longitudinal axis. The inflation lumen defines an inlet aperture and an outlet aperture and is configured to provide a fluidic connection from the inlet aperture of the inflation lumen to a balloon. The plug lumen defines an inlet aperture and an outlet aperture and is configured to deliver a plug. The microcatheter lumen defines an inlet aperture and an outlet aperture and is configured to house a microcatheter.

### RELATED APPLICATION

The present application claims the benefit of U.S. Provisional Application No. 62/167,944 filed May 29, 2015, which is hereby incorporated herein in its entirety by reference.

### TECHNICAL FIELD

Embodiments relate to surgical devices, and in particular to catheters having multiple lumens to enable several functions, such as inflating a balloon in the body, delivering a sclerosing agent, and delivering a plug.

### BACKGROUND

Conventional systems for treating various vascular conditions often include catheters configured to facilitate a variety of functions. These catheters can be routed through the vascular system from a remote incision to the site where a treatment is needed. For example, a catheter can be introduced to the vascular system at any convenient location, then routed through the vascular system to the heart, brain, or other areas that may require treatment. This prevents the need to directly access the site, by opening the chest cavity or cranium, which can be more complicated and/or less safe than a treatment delivered by catheter.

These catheters can provide any of a number of treatments. For example, catheters can be used to route a fluid, such as a liquid embolic agent or a sclerosing agent to the site. Other catheters can be used to deliver mechanical occlusion devices, such as plugs, to the site. Still other catheters can be used to provide temporary stabilization or blocking, such as by way of a balloon. On occasion, these types of catheters can be used together simultaneously or serially, by routing several catheters each having its own function to the region. However, such catheter bundles must be controlled to retain the desired relative locations within the vasculature, which can be quite difficult and require a high level of skill on the part of a surgeon using an imaging system.

Gastric varices are one type of disorder that can be treated using catheters routed to the appropriate part of the vasculature, rather than by direct surgical access. Gastric varices can be caused by a buildup of pressure in the vein. Due to the precise timing of each step in the treatment process, and the relative precision required in positioning the various catheters, however, the treatment of gastric varices using catheters can require a high amount of skill and time. Improper placement of the catheters relative to one another in the area to be treated can cause embolizing or sclerosing agents to reach areas such as the lungs or heart, which can cause harm to the patient.

Properly treating gastric varices can require, for example, a catheter that is capable of temporarily blocking blood flow (e.g., a balloon catheter), a catheter having a device that blocks off at least a portion of the flow path permanently to treat the varix (e.g., a catheter that delivers a plug), and a catheter that can seal the plug in the appropriate position in a varix after the catheter has been placed, such as by delivery of a sclerosing or embolizing agent.

As such, there is a need for a solution that allows for the catheters that accomplish each of these functions to be quickly and accurately positioned relative to one another for the treatment of gastric varices or other conditions within human vasculature.

### SUMMARY

According to a first embodiment, a catheter system comprises a catheter defining a longitudinal axis. The catheter includes an inflation lumen, a plug lumen, and a microcatheter lumen, each defined by the catheter and extend substantially parallel to the longitudinal axis. The inflation lumen is associated with an inlet aperture and an outlet aperture. The plug lumen is associated with an inlet aperture and an outlet aperture. The microcatheter lumen is associated with an inlet aperture and an outlet aperture. The catheter system further comprises a balloon arranged adjacent to the outlet aperture of the inflation lumen and defining a plenum, wherein the plenum is fluidically coupled to the inlet aperture of the inflation lumen. The catheter system further comprises a microcatheter disposed within the microcatheter lumen.

According to a second embodiment, a method includes providing a catheter, and forming multiple lumens in the catheter. The lumens comprise an inflation lumen, a plug lumen, and a microcatheter lumen. Each of the inflation lumen, the plug lumen, and the microcatheter lumen defines an inlet aperture and an outlet aperture. The method further comprises providing a balloon at the outlet aperture of the inflation lumen. The method further comprises providing a plug in the plug lumen.

According to a third embodiment, a multi-lumen catheter defines a longitudinal axis. The catheter comprises an inflation lumen defined by the catheter and extending substantially parallel to the longitudinal axis, wherein the inflation lumen defines an inlet aperture and an outlet aperture and is configured to provide a fluidic connection from the inlet aperture of the inflation lumen to a balloon. The catheter further comprises a plug lumen defined by the catheter and extending substantially parallel to the longitudinal axis, wherein the plug lumen defines an inlet aperture and an outlet aperture and is configured to deliver a plug. The catheter further comprises a microcatheter lumen defined by the catheter and extending substantially parallel to the longitudinal axis, wherein the microcatheter lumen defines an inlet aperture and an outlet aperture and is configured to house a microcatheter.

### BRIEF DESCRIPTION OF THE DRAWINGS

FIG. 1A depicts a multi-lumen catheter according to an embodiment.

FIG. 1B depicts a microcatheter extending from an end of the multi-lumen catheter of FIG. 1A.

FIG. 2 is a cross-sectional view of the catheter of FIGS. 1A-1B, taken along line 2-2.

FIG. 3 is an end view of the catheter of FIGS. 1A-1B, from a first end.

FIG. 4 is an end view of the catheter of FIGS. 1A-1B, from a second end.

FIGS. 5A and 5B are perspective views of a catheter according to an embodiment.

FIGS. 6A and 6B are perspective views of a catheter hub according to an embodiment.

While embodiments are amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The challenges previously described with respect to contemporaneous use of multiple catheters, each performing a separate function, can be mitigated according to embodiments of the catheters and methods described herein. A multi-lumen catheter can include one or more lumens for each desired treatment or task. For example, in some embodiments a three-lumen catheter includes one lumen that connects the operator's end of the catheter to a balloon, a second lumen that contains a plug to be placed in the vasculature, and a third lumen that can be used to route a microcatheter, for example, to deliver a sclerosing or embolizing agent. In this way, only one multi-lumen catheter need be routed to the area of the vasculature that is being treated. Because the output apertures of the multi-lumen catheter are set in a fixed spatial relationship with one another, the task of the operator or surgeon is much less difficult and is also more predictable. Combining all of the necessary lumens to perform Balloon-Occluded Retrograde Transvenous Obliteration (BRTO) safely through one venous puncture increases the chances that the position of the catheter stays stable during the delivery of the vascular plug as well as coils and sclerosing agent required to shut down the gastric varices.

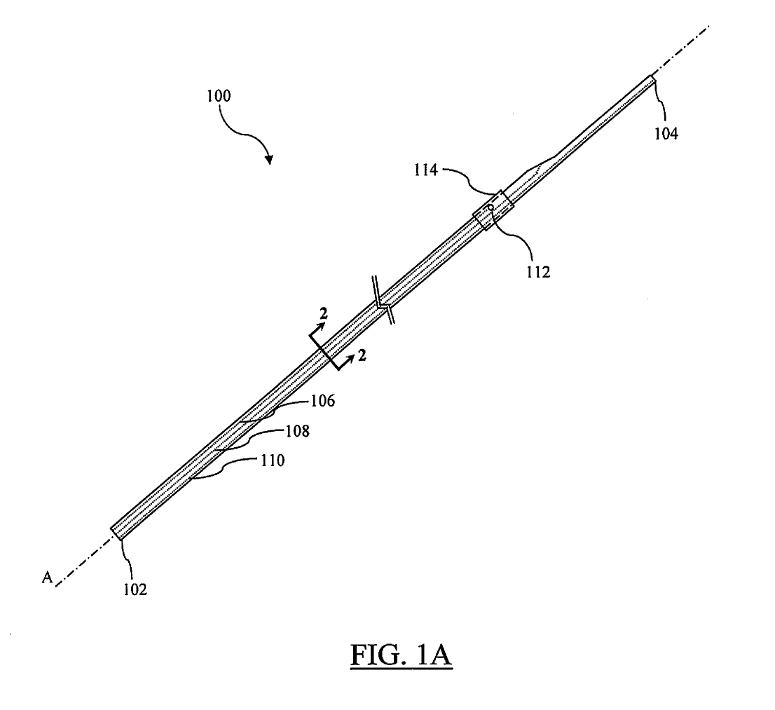

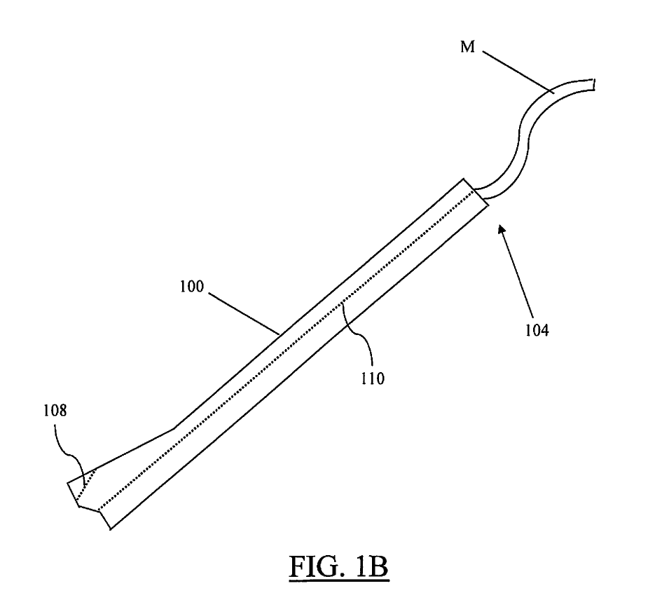

FIG. 1A depicts a multi-lumen catheter 100, according to an embodiment. Multi-lumen catheter 100 extends between a first end 102 and a second end 104 along longitudinal axis A. Because multi-lumen catheter 100 is often flexible and bendable to travel through vasculature, longitudinal axis A need not be rectilinear, and can in fact vary in both direction and curvature during use of multi-lumen catheter 100. Multi-lumen catheter 100 can be substantially longer than it is wide in some embodiments as indicated by the broken lines in FIG. 1A, such as about 60 centimeters to about 100 centimeters in embodiments, in order to reach parts of the vasculature that are relatively distant from the point of entry of catheter 100.

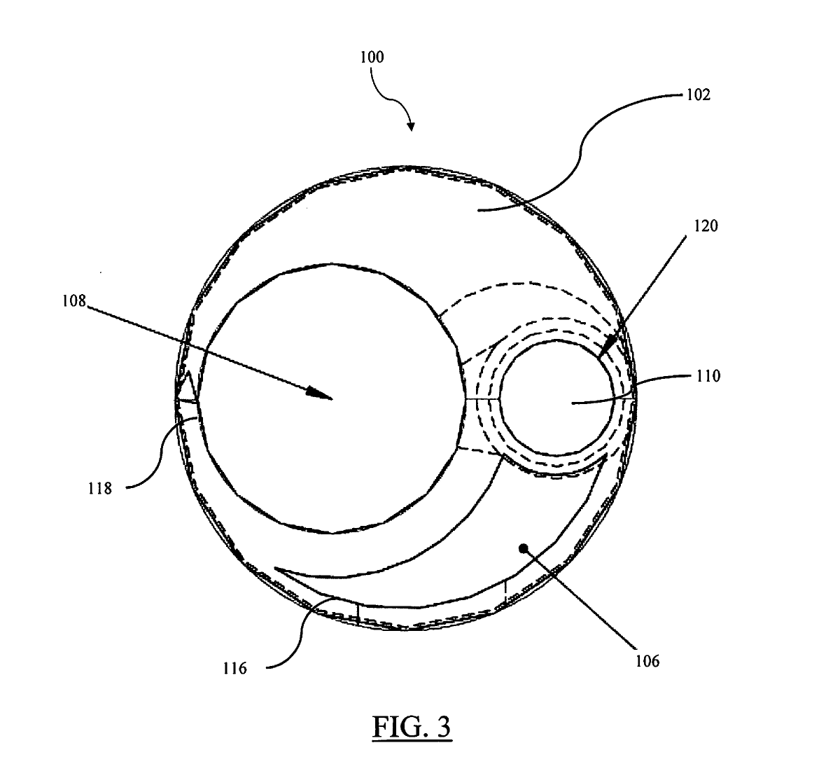

Multi-lumen catheter 100 defines inflation lumen 106, plug lumen 108, and microcatheter lumen 110 in the embodiment shown in FIG. 1A. The three lumens 106, 108, and 110 extend through the body of the multi-lumen catheter 100 substantially parallel to longitudinal axis A, along paths as indicated by the dotted lines in FIG. 1A. In the embodiment shown in FIG. 1A, each of the lumens 106, 108, and 110 defines an inlet aperture (apertures 116, 118, and 120, respectively, as shown in FIG. 3) in multi-lumen catheter 100 at first end 102. Each of the lumens 106, 108, and 110 further defines an outlet aperture, and these outlet apertures are located at different locations along longitudinal axis A.

In the embodiment shown in FIG. 1A, outlet aperture 112, the outlet of inflation lumen 106, is shown. Aperture 112 is located adjacent to balloon 114. Balloon 114 can be, for example, an elastomeric material mounted on multi-lumen catheter 100, coaxially about longitudinal axis A. Pressurized fluid can be routed from first end 102 through inflation lumen 106, where it exits at aperture 112 to inflate balloon 114.

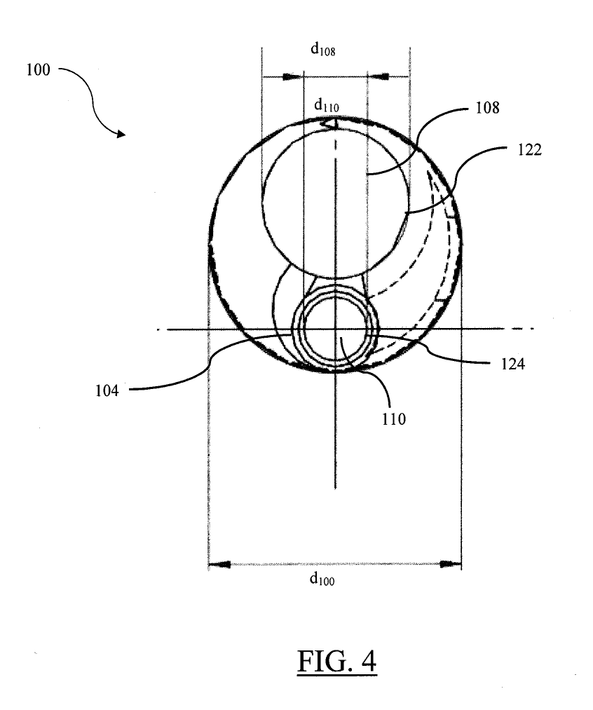

In the embodiment shown in FIGS. 1A and 4, inflation lumen 106 defines the outlet aperture 112 relatively closest to first end 102; microcatheter lumen 110 defines the outlet aperture 124 relatively closest to second end 104; and plug lumen 108 defines outlet aperture 122 that is positioned between them along axis A. This particular arrangement of outlet apertures 112, 122, and 124 can be advantageous where multi-lumen catheter 100 is used in a retrograde approach, such as for BRTO. In a retrograde approach, balloon 114 will be furthest downstream in the blood flow, plug lumen 108 terminates upstream of balloon 114, and the microcatheter lumen 110 terminates furthest in the upstream direction. Thus, in use for treatment of a gastric varix, for example, balloon 114 can be inflated most proximate to the location where the varix drains to an artery, a plug can be inserted in the varix via plug lumen 108, for example by pushing a plug through plug lumen 108 using a ramrod or screw, and a microcatheter can deliver a sclerosing or embolizing agent upstream of both the plug and the inflated balloon. There is very little chance of a sclerosing agent bypassing both the plug and the inflation balloon to enter the bloodstream and cause a problem by affecting other areas of the vasculature such as the lungs.

Other arrangements of outlet apertures 112, 122, and 124 can be used in other embodiments. In some embodiments, the distance between outlets 112, 122, and 124 can be between about 1 centimeter and about 15 centimeters, or more particular between about 5 centimeters and about 9 centimeters. In other embodiments, the order in which outlet apertures 112, 122, and 124 are positioned along catheter 100 can be rearranged, such as for treatment in an antegrade rather than retrograde approach, or for treatment of a different vascular disorder, or for treatment of non-vascular disorders. It can be appreciated that the spacing and order of outlet apertures 112, 122, and 124 along catheter 100 can be selected to suit a variety of different procedures, each procedure potentially having a different optimal spacing as well as ordering of outlet apertures 112, 122, and 124. It can also be appreciated that even where the spacings, orderings, or even the number of outlets in alternative catheters varies from that which is shown with respect to catheter 100 of FIG. 1A, the benefits of the fixed spatial relationship are maintained. Likewise, the beneficial reduction in complexity and operator skill required, due to the reduction in the number of parallel catheters, is also maintained in such alternative embodiments.

FIG. 1B is a closeup view of second end 104 of catheter 100 previously discussed with respect to FIG. 1A. In addition to the components previously discussed, FIG. 1B depicts a microcatheter M extending from second end 104. In the embodiment shown in FIG. 1B, microcatheter M extends beyond second end 104. In use, microcatheter M need not extend beyond second end 104. Rather, microcatheter M could be surrounded by microcatheter lumen 110 all the way to the terminus of microcatheter M.

In an alternative embodiment, a sclerosing or embolizing material can be delivered directly via microcatheter lumen 110 (i.e., without actually using a microcatheter). Furthermore, various alternative substances, such as antibacterial or antiseptic agents, or anaesthetics, among others, can be delivered via microcatheter lumen 110. In fact, in some alternative embodiments, multi-lumen catheter 100 can include more than one microcatheter lumen 110 or other lumen (not shown) to deliver fluids to a desired site.

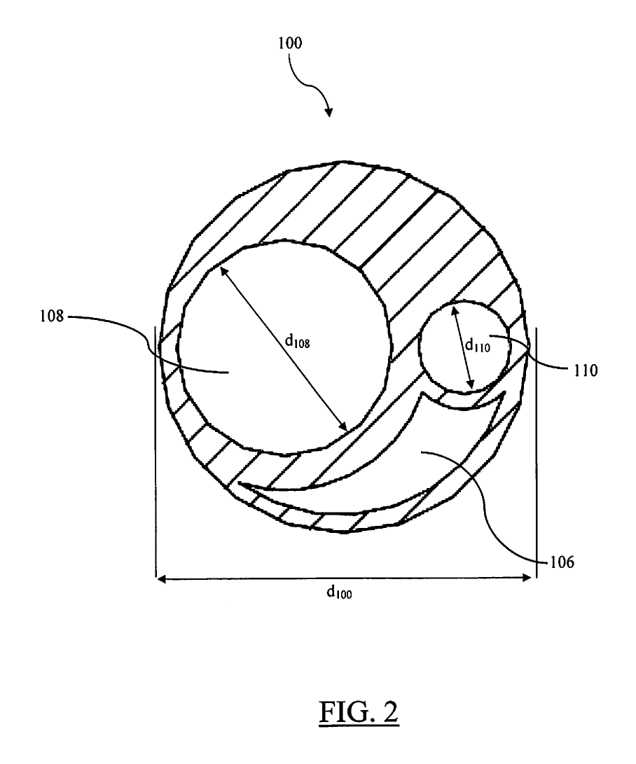

A cross-section of multi-lumen catheter 100 of FIGS. 1A and 1B is shown in FIG. 2, along cross-section 2-2 of in FIG. 1A. As shown in FIG. 2, inflation lumen 106 is irregularly shaped, whereas plug lumen 108 and microcatheter lumen 110 have a substantially circular cross-section. In other words, inflation lumen 106 need not have a circular cross-section along the plane perpendicular to the axis A shown in FIG. 1A. This is because, in the embodiment shown in FIGS. 1A-2, a substantially cylindrical plug is delivered via plug lumen 108 and a substantially cylindrical microcatheter can be routed through microcatheter lumen 110. In contrast, inflation lumen 106 routes a pressurized fluid such as saline or air, which can pass through any shaped lumen. Thus, inflation lumen 106 can be shaped to maximize its cross-sectional area while also retaining sufficient wall thickness between inflation lumen 106 and the adjacent plug lumen 108, microcatheter lumen 110, and even the outer wall of the multi-lumen catheter 100. In other embodiments, each of the lumens 106, 108, 110, or additional lumens not shown in the embodiment depicted in FIGS. 1A and 1B, can have any of a variety of cross-sections.

FIG. 2 depicts the diameters of the lumens 108 and 110 having circular cross-sections. In particular, diameter d₁₀₈ is the diameter of plug lumen 108, and diameter d₁₁₀ is the diameter of microcatheter lumen 110. These diameters can be sized to facilitate their previously-mentioned functions; that is, plug lumen diameter d₁₀₈ can be sized to permit travel of a plug (not shown) through plug lumen 108, and microcatheter diameter d₁₁₀ can be sized to permit travel of a microcatheter through microcatheter lumen 110. In one embodiment, plug lumen diameter d₁₀₈ is about 0.23 cm (0.091 inches), corresponding to a 7 French lumen size for plug lumen 108. In one embodiment, diameter d₁₁₀ is about 0.10 cm (0.039 inches), corresponding to a 3 French lumen size for microcatheter lumen 110. In alternative embodiments, these sizes can vary. For example, a relatively smaller or larger plug may be desired for different varices or any other type of vascular disorder, and in those cases a relatively smaller or larger French size can be used. In some embodiments, the lumens can be slightly larger than a standard French size, to accommodate a microcatheter or plug of that French size without interference or friction.

Diameters d₁₀₈ and d₁₁₀ determine, at least in part, the overall diameter of the catheter 100. As shown in FIG. 2, catheter 100 has an overall diameter d₁₀₀ of about 0.396 cm (0.156 inches). In other embodiments this overall diameter can be relatively larger or smaller, but in general it will be sufficiently large to accommodate each of lumens 106, 108, and 110. Additionally, the overall diameter can include sufficient wall thickness separating lumens 106, 108, and 110 from one another, as well as from the exterior of catheter 100.

In alternative embodiments, the plug that is delivered via plug lumen 108 need not have a circular cross-section. In that case, plug lumen 108 could have a different cross-section configured to match that of the plug. Likewise, if a microcatheter (such as microcatheter M of FIG. 1B) is used that does not have a cylindrical cross-section, microcatheter lumen 110 need not have a circular cross-section either. Various sizes and shapes of inflation lumens, microcatheter lumens, and/or plug lumens can be combined in various embodiments similar to multi-lumen catheter 100, and these lumens can be ‘packed’ within catheter 100 so that the overall diameter d₁₀₀ remains relatively small.

For example, in the embodiment shown in FIG. 2, inflation lumen 106 need not have a circular cross-section, and a relatively large cross-section reduces pressure drop across the length of catheter 100. Thus, it can be beneficial to employ an irregularly shaped inflation lumen 106, which gives a relatively large cross-sectional area without increasing the overall diameter d₁₀₀.

FIG. 3 shows catheter 100, as previously described with respect to FIGS. 1A, 1B, and 2, from first end 102. In addition to the features previously described with respect to FIGS. 1A, 1B, and 2, FIG. 3 also shows inlet apertures 116, 118, and 120. Inlet aperture 116 is defined by inflation lumen 106, and allows for ingress of a fluid such as air or saline that can inflate balloon 114, as previously described with respect to FIG. 1A. Inlet aperture 118 is defined by plug lumen 108, and inlet aperture 118 allows for a plug or other material to be inserted into that lumen 108. Likewise, inlet aperture 120 is defined by microcatheter lumen 110, and inlet aperture 120 allows for a microcatheter (M, FIG. 1B) to be inserted into that lumen 110.

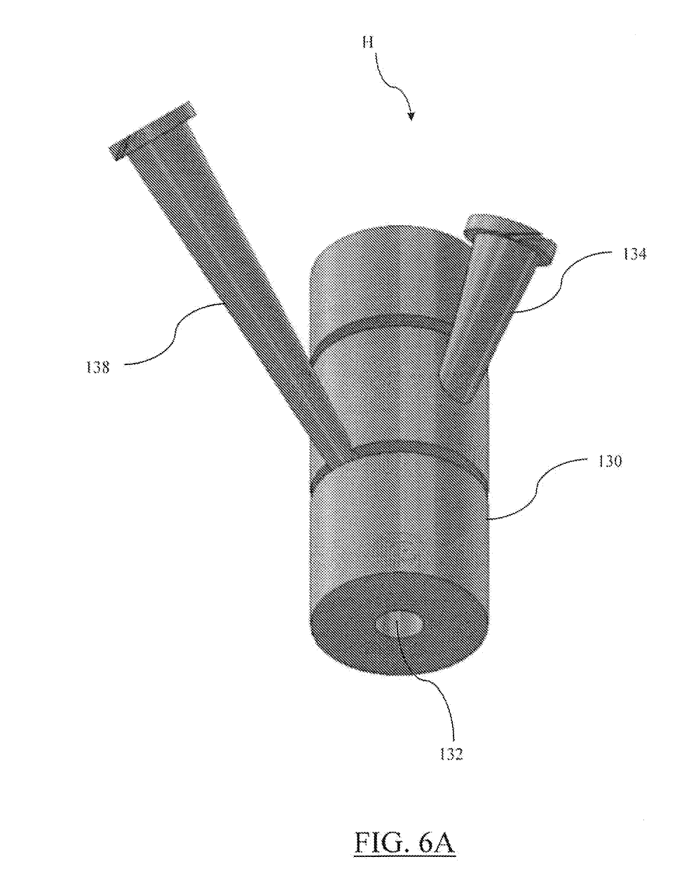

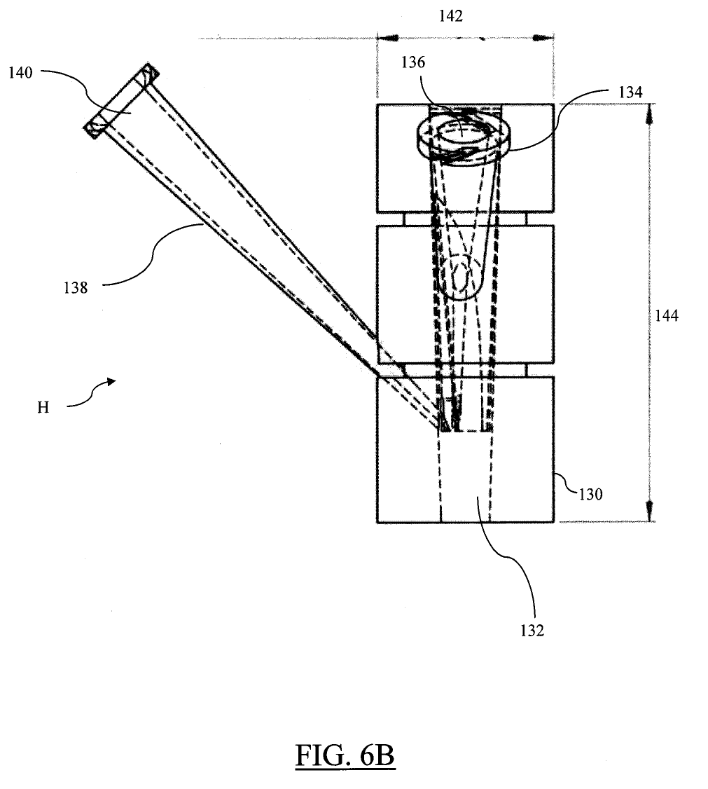

As described in more detail with respect to FIG. 6, catheter 100 is adapted to receive inputs such as pressurized fluid, a plug, or a microcatheter, for example, which are received at inlet apertures 116, 118, and 120. Therefore, first end 102 can be configured to be positioned inside a catheter hub during use, as described in more detail with respect to FIGS. 6A and 6B. Various known structures can be used to interface between the first end 102 of catheter 100 and these inputs.

FIG. 4 shows catheter 100, as previously described with respect to FIGS. 1A-3, from the second end 104. In addition to the features previously described with respect to FIGS. 1A-3, FIG. 4 also shows outlet apertures 122 and 124, which are associated with plug lumen 108 and microcatheter lumen 110, respectively.

In the embodiment shown in FIG. 4, only two of these outlet apertures, 122 and 124, are visible, and the outlet aperture 112 associated with inflation lumen 106 is not seen. This is because, as shown in FIG. 1A, inflation lumen 106 defines an outlet aperture 112 that allows egress substantially perpendicular to the axis A, rather than parallel to axis A. This facilitates the expansion of balloon 114 radially outwards from the catheter 100, whereas the plug lumen 108 and microcatheter lumen 110 are configured to deliver a plug and a microcatheter, respectively, in a forward axial direction i.e., along axis A as shown in FIG. 1A. In alternative embodiments, other types and shapes of balloons could be employed which inflate to various sizes and shapes. These alternative balloons need not always be symmetric about axis A, in some embodiments.

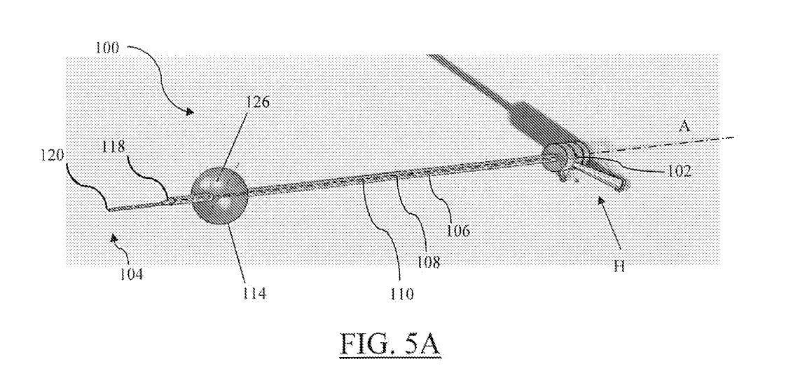

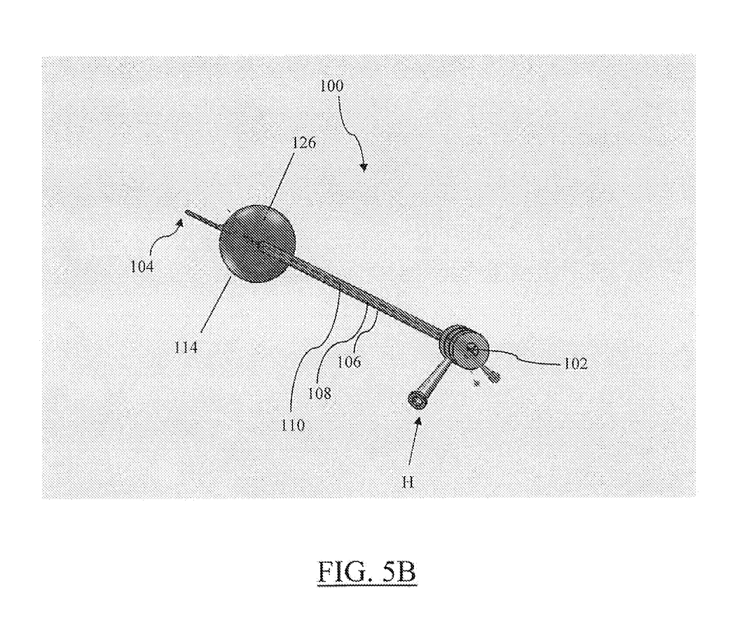

FIGS. 5A and 5B show catheter 100 and hub H. As shown in FIGS. 5A-5B, balloon 114 is inflated, and first end 102 is positioned within the hub H.

By inflating balloon 114, a plenum 126 expands radially outward from axis A. Plenum 126 is a region of pressurized fluid located between balloon 114 and catheter 100. In one use of catheter 100 and hub H, catheter 100 can be routed through hub H, inserted into a patient, and second end 104 can be routed to a site for treatment (such as a gastric varix). Balloon 114 can be inflated once second end 104 is in a desired position. For example, the desired position could be the location in which balloon 114 blocks blood flow either into or out of the varix. Balloon 114 can be inflated until there is an interference fit between balloon 114 and a surrounding portion of the vasculature.

Once balloon 114 is inflated, the other lumens in catheter 100 can be utilized to treat the varix or perform some other procedure. For example, in the embodiment shown in FIGS. 5A-5B, plug lumen 108 can deliver a plug at outlet aperture 118, which is disposed between balloon 114 and second end 104. Once balloon 114 is inflated, the level of blood flow around outlet aperture 118 can be significantly reduced or eliminated, simplifying the process of accurately placing the plug. Furthermore, because catheter 100 is held in an interference fit with the varix by balloon 114, there can be lower relative movement between outlet aperture 118 and the varix as well, which also simplifies the plug placement process.

Similarly, during use, outlet aperture 120 of microcatheter lumen 110 can deliver a sclerosing agent, embolizing agent, or some other material, to the vasculature. Balloon 114 can prevent such materials from dispersing, by reducing or eliminating blood flow out of the site being treated. This not only enhances the effectiveness of the dose that is administered (because it remains at the site), but also prevents any undesirable effects from, for example, the sclerosing agent leaving the site and flowing to the lungs or another area where it could cause injury.

FIGS. 6A-6B depict hub H in more detail. Hub H includes a body portion 130 defining a common line 132, inflation lumen arm 134 defining inflation lumen line 136, and microcatheter lumen arm 138 defining microcatheter lumen line 140.

Common line 132 travels through the body portion 130, and a catheter such as catheter 100 described with respect to FIGS. 1A-5B can be positioned in the common line 132. As previously described, first end 102 of catheter 100 includes inlet apertures 116, 118, and 120 associated with each of lumens 106, 108, and 110, respectively. Inlet apertures 116, 118, and 120 are configured to receive the various devices and/or substances that are routed through catheter 100. Hub H facilitates the ingress of these devices and/or substances. In particular, in the embodiment shown in FIGS. 6A and 6B, inflation lumen line 136 is configured to carry a pressurized fluid to inlet aperture 116 of inflation lumen 106, and microcatheter lumen line 140 is configured to route a microcatheter to inlet aperture 120 of microcatheter lumen 110.

In alternative embodiments, such as those in which fewer or more lumens are present in the catheter, a different hub can be used which has fewer or more arms. Furthermore, depending on the type of material or device being routed to the catheter, differently angled or sized arms can be used, for example.

FIG. 6B further depicts first hub dimension 142 and second hub dimension 144. First hub dimension can be about 1.46 cm (0.575 inches), for example. Second hub dimension 144 can be about 3.467 cm (1.365 inches), for example. In alternative embodiments, hub dimensions 142 and 144 could be larger or smaller in order to accommodate different materials or devices being routed to catheter 100.

Various embodiments of systems, devices and methods have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A catheter system comprising:
a catheter defining a longitudinal axis;
an inflation lumen, a plug lumen, and a microcatheter lumen each defined by the catheter and extend substantially parallel to the longitudinal axis, wherein:
the inflation lumen is associated with an inlet aperture and an outlet aperture;
the plug lumen is associated with an inlet aperture and an outlet aperture; and
the microcatheter lumen is associated with an inlet aperture and an outlet aperture;
a balloon arranged adjacent to the outlet aperture of the inflation lumen and defining a plenum, wherein the plenum is fluidically coupled to the inlet aperture of the inflation lumen; and
a microcatheter disposed within the microcatheter lumen.

2. The catheter system of claim 1, wherein the plug lumen comprises a 7 French passage.

3. The catheter system of claim 1, wherein the microcatheter lumen comprises about a 3 French passage.

4. The catheter system of claim 3, wherein the microcatheter has a size of 3 French.

5. The catheter system of claim 1, wherein the balloon is configured to extend radially about the longitudinal axis.

6. The catheter system of claim 1, wherein a cross-sectional shape of the inflation lumen along a plane orthogonal to the axis is non-circular.

7. The catheter system of claim 1, further comprising a catheter hub configured to receive a first end of the catheter comprising the inlet apertures.

8. The catheter system of claim 7, wherein at least one of the plurality of apertures associated with each of the inflation lumen, the plug lumen, and the microcatheter lumen is disposed within a common line defined by the hub.

9. The catheter system of claim 8, wherein the microcatheter extends through an aperture associated with the microcatheter lumen at an end of the catheter opposite from the hub.

10. The catheter system of claim 8, wherein the microcatheter does not extend through an aperture associated with the microcatheter lumen at an end of the catheter opposite from the hub.

11. A method comprising:
providing a catheter;
forming multiple lumens in the catheter, wherein the lumens comprise an inflation lumen, a plug lumen, and a microcatheter lumen, each of the inflation lumen, the plug lumen, and the microcatheter lumen defining an inlet aperture and an outlet aperture; and
providing a balloon at the outlet aperture of the inflation lumen.

12. The method of claim 11, wherein the balloon is configured to form an interference fit with a vasculature.

13. The method of claim 12, wherein the vasculature comprises a gastric varix.

14. The method of claim 11, wherein the balloon is configured to be inflated by routing a fluid through the inflation lumen.

15. The method of claim 11, wherein the catheter is configured to be used in a retrograde approach.

16. The method of claim 15, wherein the catheter is configured to be used in balloon-occluded retrograde transvenous obliteration.

17. A multi-lumen catheter defining a longitudinal axis, the catheter comprising:
an inflation lumen defined by the catheter and extending substantially parallel to the longitudinal axis, wherein the inflation lumen defines an inlet aperture and an outlet aperture and is configured to provide a fluidic connection from the inlet aperture of the inflation lumen to a balloon;
a plug lumen defined by the catheter and extending substantially parallel to the longitudinal axis, wherein the plug lumen defines an inlet aperture and an outlet aperture and is configured to deliver a plug; and
a microcatheter lumen defined by the catheter and extending substantially parallel to the longitudinal axis, wherein the microcatheter lumen defines an inlet aperture and an outlet aperture and is configured to house a microcatheter.

18. The catheter of claim 17, wherein the plug lumen comprises about a 7 French passage.

19. The catheter of claim 17, wherein the microcatheter lumen comprises about a 3 French passage.

20. The catheter of claim 17, wherein a cross-sectional shape of the inflation lumen along a plane orthogonal to the axis is non-circular.
